(19) United States
(12) Patent Application Publication
(10) Pub. No.: US 2008/0177146 A1
(43) Pub. Date: Jul. 24, 2008

(54) Double Vision Endotracheal Tube Installation System

(76) Inventor: Tien-Sheng Chen, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/776,798

(22) Filed: Jul. 12, 2007

(30) Foreign Application Priority Data
Jan. 19, 2007 (TW) 096201171

(51) Int. Cl. A61B 1/267

(52) U.S. Cl. 600/185; 600/188; 600/190

(57) ABSTRACT

A double vision endotracheal tube installation system is disclosed, said system comprising: a laryngoscope, comprising a handgrip and a blade, wherein the blade comprises a first image-capturing unit for capturing a first image; a hollow endotracheal tube; a tracheoscope, comprising a second image-capturing unit for capturing a second image, said second image-capturing unit being wrapped in the endotracheal tube; and at least one display for displaying said first image and second image.

### BACKGROUND OF THE INVENTION

1. Field of the Invention

The present invention relates to an endotracheal tube installation system, more particularly to an endotracheal tube installation system capable of providing a dual view

2. Description of the Related Art

Endotracheal intubation is a critical means for maintaining the breathing function of a patient under general anaesthesia. In most cases, to prevent the occurrence of hypoxia, the anaesthetist must complete the intubation by inserting an endotracheal tube into the patient's trachea in a very short period of time to provide oxygen thereinto promptly. Therefore, it is extremely important for anaesthetists to perform the intubation efficiently.

Practically, to intubate quickly, most anaesthetists use a laryngoscope as a means to observe the condition of a patient's upper airway. Please refer to FIG. 1. An early laryngoscope 10 mainly consists of a handgrip 20 and a blade 30, wherein the blade may further comprise a first image-capturing unit 40 for tracking the condition in the patient's upper airway. In use, the anaesthetists may have the patient lay face up and raise the patient's jaw first; after that, they may depress the tongue base with the blade 30 by holding the handgrip 20 so as to raise the epiglottis cartilage. Then the image captured by the first image-capturing unit 40 may be used to facilitate the intubation.

However, the aforementioned approach fails to fully satisfy practical needs. Refer now to FIG. 2 for an illustrative diagram showing the application of a laryngoscope used in prior arts to perform intubation. First, it should be appreciated that, after an endotracheal tube 70 has been delivered into the patient's upper airway, the endotracheal tube 70 will inevitably obstruct the view of the first image-capturing unit 40. Thus, neither the condition of the patient's upper airway nor the position of the trachea can be known by the anaesthetists, with the result that they can only rely on personal experience and skill during the intubation. In addition, since the anatomical structures of different patients vary significantly, an intubation system applying only one image pickup apparatus may fail to satisfy the needs of different cases. Accordingly, it is important to provide an endotracheal tube installation system capable of providing a better view.

### SUMMARY OF THE INVENTION

It is an objective of the present invention to provide an endotracheal tube installation system with an enhanced view.

It is another objective of the present invention to provide an endotracheal tube installation system which has two image pickup apparatuses. Said endotracheal tube installation system is capable of transmitting images wirelessly so as to increase the precision and efficiency of the intubation.

To attain these goals, this invention provides an endotracheal tube installation system, comprising: a laryngoscope, comprising a handgrip and a blade, wherein the blade comprises a first image-capturing unit for capturing a first image; a hollow endotracheal tube; a tracheoscope, comprising a second image-capturing unit for capturing a second image, said second image-capturing unit being wrapped in the endotracheal tube; and at least one display for displaying said first image and second image.

By the use of the system of the present invention, users may use two individual image-capturing units to observe the condition of a patient's upper airway without encountering the problem of image obstruction. In use, users may observe the image captured by the first image-capturing unit and then deliver into the patient's mouth the endotracheal tube and the second image-capturing unit in the endotracheal tube. When the view of the first image-capturing unit is obstructed by the endotracheal tube, the second image-capturing unit may be adopted to provide images from deep in the airway to allow location of the position of the trachea. After the position of the trachea is determined, the endotracheal tube may be pushed forward into the trachea and the tracheoscope may be drawn out of the patient to complete the intubation.

It should be noted that the transmission of the image captured by the two image-capturing units may be done by wireless means, for example, by the installation of an emitter and a receiver. Since the technology for the wireless image transmission is already known, further elaboration is omitted accordingly.

Also, the display used in the invention may be two separate monitors displaying the first image and the second image individually; alternatively, the display may also be a single monitor displaying the first image and the second image simultaneously or by manual switch. Thereby, users may get a better understanding of the patient's upper airway and, more importantly, carry out the intubation with higher efficiency and precision.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 illustrates a laryngoscope used in prior arts.

FIG. 2 is an illustrative diagram showing the application of a laryngoscope used in prior arts to perform intubation.

FIG. 3A is an illustrative diagram showing the second image-capturing unit of the present invention encompassed by an endotracheal tube.

FIG. 3B is an illustrative diagram showing the application of the endotracheal tube installation system of the present invention to perform intubation.

FIG. 4 is a flowchart showing the steps for applying the endotracheal tube installation system of the present invention.

FIGS. 5A to 5D are illustrative diagrams showing the steps for applying the endotracheal tube installation system of the present invention.

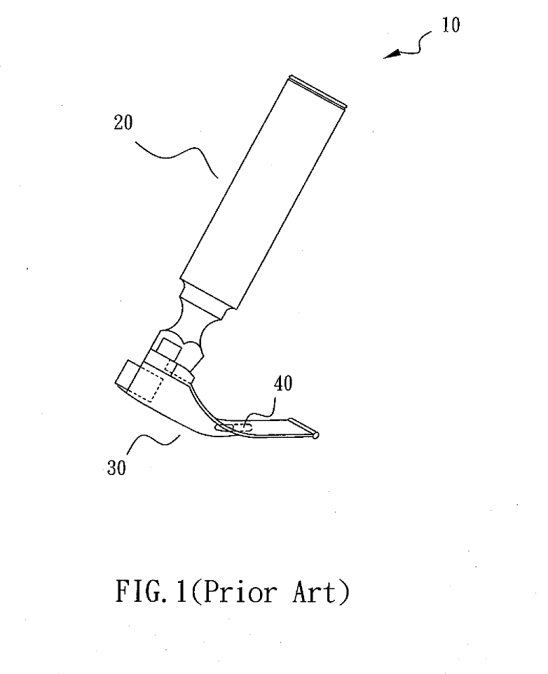

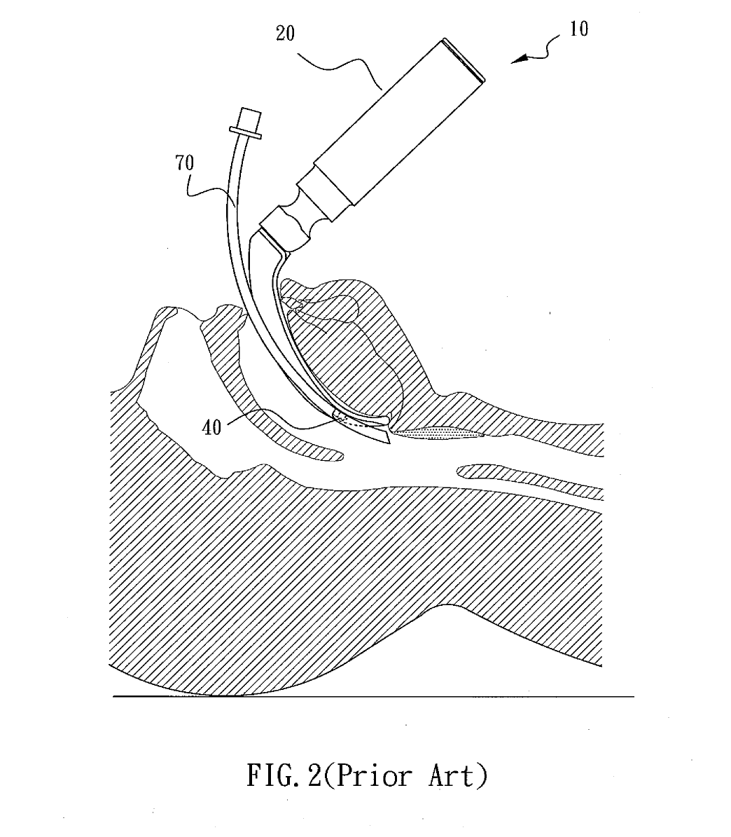

### DETAILED DESCRIPTION OF THE PREFERRED EMBODIMENT

To make this invention more understandable to examiners, several preferred embodiments are disclosed and described hereafter.

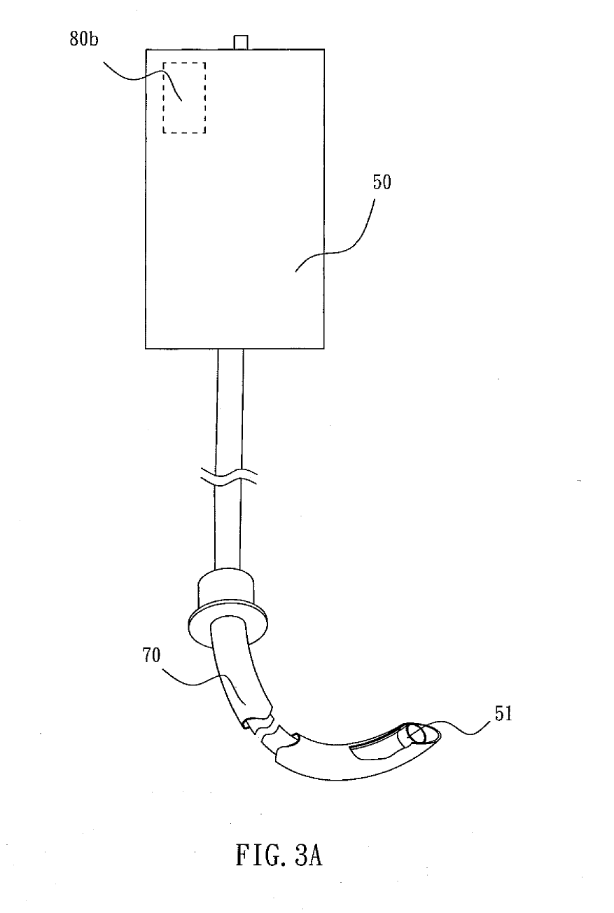

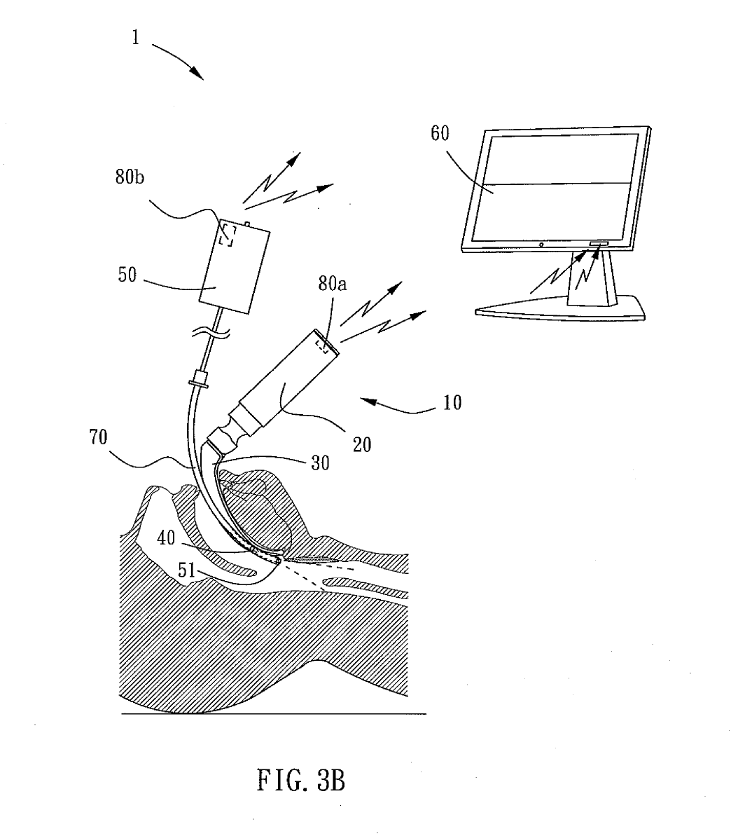

Please refer to FIGS. 3A and 3B, wherein FIG. 3A is an illustrative diagram showing the second image-capturing unit of the present invention encompassed by an endotracheal tube, and FIG. 3B is an illustrative diagram showing the application of the endotracheal tube installation system of the present invention to perform intubation. The endotracheal tube installation system 1 of the present invention mainly comprises a laryngoscope 10, a tracheoscope 50, an endotracheal tube 70, and at least one display 60. The laryngoscope 10 comprises a handgrip 20, a blade 30 connected to the handgrip 20, and a first image-capturing unit 40 installed on the blade 30 for capturing a first image. The endotracheal tube 70 is a hollow tube encompassing the second image-capturing unit 51 of the tracheoscope 50, which is used for capturing a second image. In addition, to enable the first and the second images to be transmitted wirelessly to the display 60, the endotracheal tube installation system 1 may further comprise emitters 80a and 80b installed on the laryngoscope 10 and the tracheoscope 50, respectively. Emitters 80a and 80b are connected electrically to the first image-capturing unit 40 and the second image-capturing unit 51, respectively, for transmitting the first and the second images to the display 60 by wireless means.

What should be noted is that the display 60 may be two individual monitors showing the first and the second images separately; alternatively, the display 60 may also be a single monitor displaying the first image and the second image simultaneously or by manual switch. Furthermore, even though it is shown that the display 60 is installed externally, as shown in FIG. 3B, the display 60 may also be installed on the laryngoscope 10 or the tracheoscope 50 without interfering with the operability.

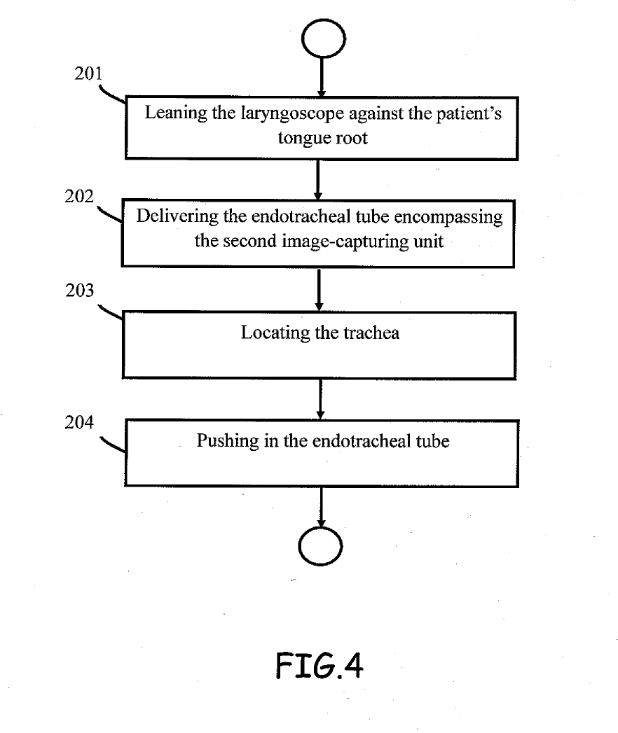

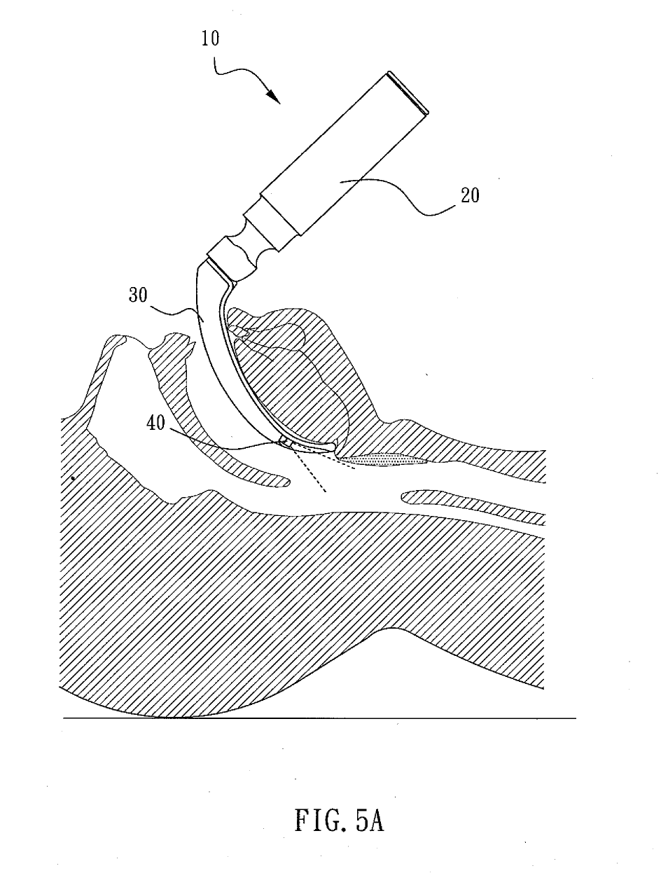

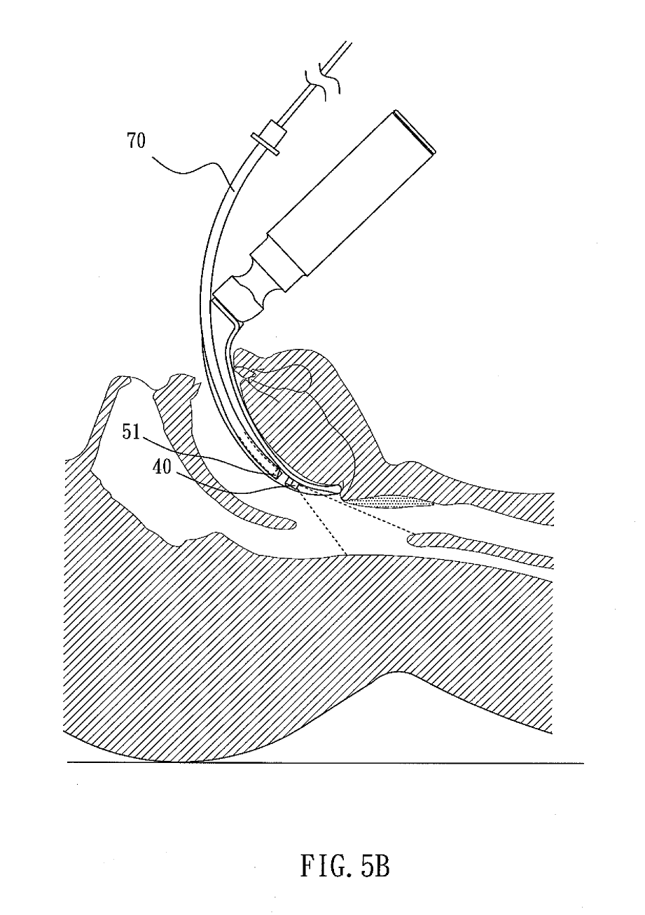

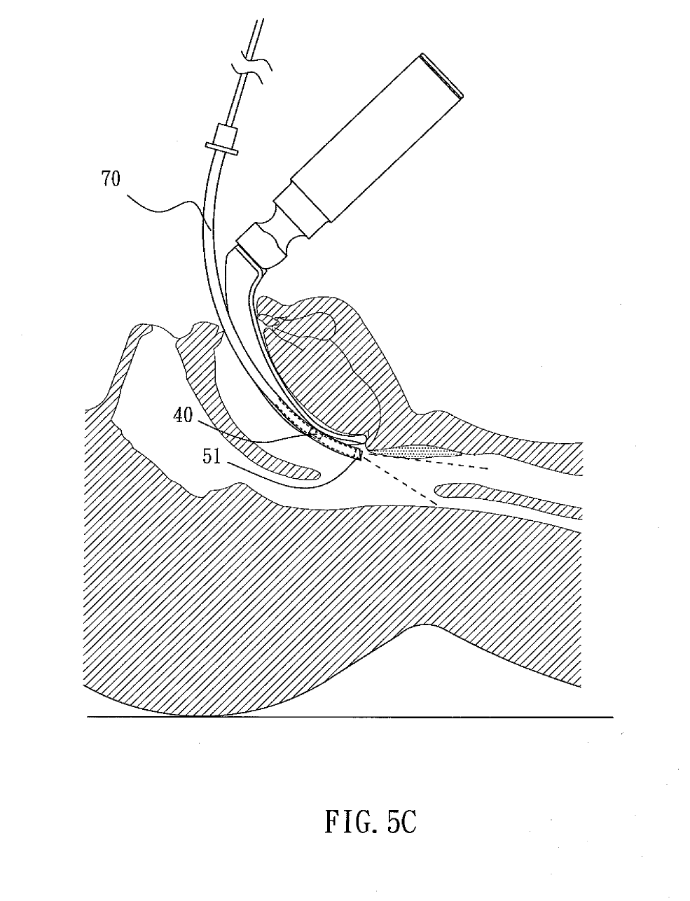

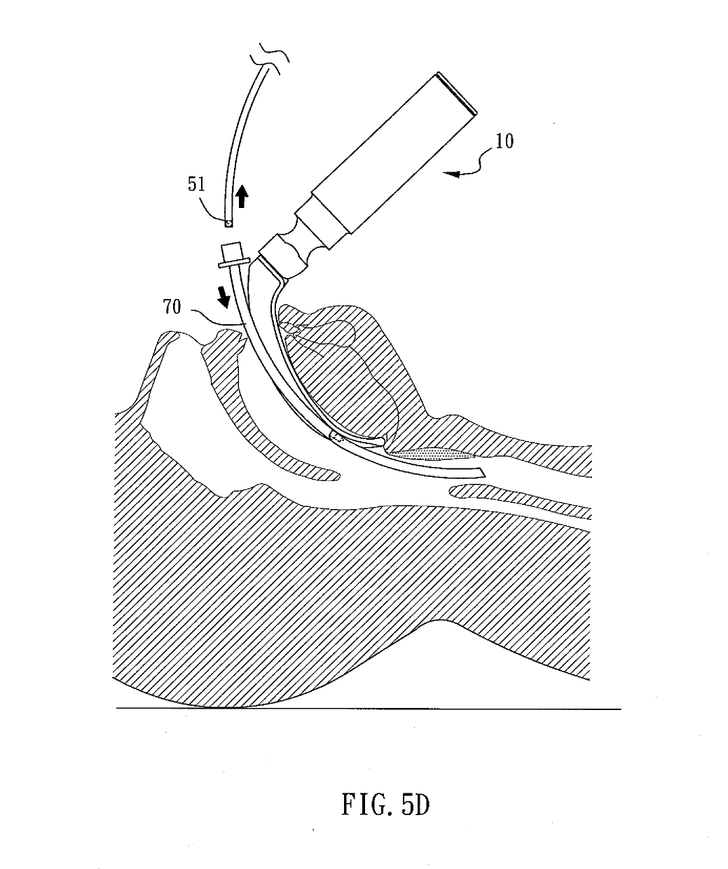

Refer now to FIG. 4 for a flowchart showing the steps for applying the endotracheal tube installation system 1 of the present invention. Meanwhile, refer to FIGS. 5A to 5D for illustrative diagrams showing the steps for applying the endotracheal tube installation system 1 of the present invention.

201: A user presses the laryngoscope 10 against the patient's tongue base.

As shown in FIG. 5A, to obtain a better view, a user may have a patient lay face up and raise the patient's jaw first; after that, the user may depress the tongue base with the blade 30 by holding the handgrip 20 of the laryngoscope 10 so as to raise the epiglottis cartilage. During the process, the first image-capturing unit 40 installed on the blade 30 may capture the first image of the patient's upper airway.

202: The user delivers the endotracheal tube 70 encompassing the second image-capturing unit 51 into the patient's upper airway.

As shown in FIG. 5B, from the use of the first image, the user may gain a general idea of the condition of the patient's upper airway. Thus, the user may then deliver the endotracheal tube 70 together with the second image-capturing unit 51 wrapped therein into the patient's upper airway. Under the guidance of the first image, the user may more precisely install the endotracheal tube 70 and the second image-capturing unit 51, and the randomness of unguided operation may be reduced. So far, the endotracheal tube 70 has not obstructed the view of the first image-capturing unit 40, so the user relies mainly on the first image.

203: The user begins to locate the trachea.

As shown in FIG. 5C, the user continues delivering the endotracheal tube 70 and the second image-capturing unit 51 beyond the first image-capturing unit 40, and, at the same time, the endotracheal tube 70 may obstruct the view of the first image-capturing unit 40. Accordingly, the user may turn to the second image, which is captured by the second image-capturing unit 51. Under the guidance of the second image, the user may precisely locate the trachea.

204: The user pushes the endotracheal tube 70 into the trachea.

As shown in FIG. 5D, when the user delivers the endotracheal tube 70 and the second image-capturing unit 51 to the opening of the trachea, he/she may directly push the endotracheal tube 70 into the trachea and gradually draw back the second image-capturing unit 51; after removing the laryngoscope 10, the user may complete the intubation.

It will be understood that many other modifications can be made to the various disclosed embodiments without departing from the spirit and scope of the invention. For these reasons, the above description should not be construed as limiting the invention, but should be interpreted as merely exemplary of preferred embodiments.

What is claimed is:

1. An endotracheal tube installation system, comprising:
a laryngoscope, comprising a handgrip and a blade, wherein the blade comprises a first image-capturing unit for capturing a first image;
a hollow endotracheal tube;
a tracheoscope, comprising a second image-capturing unit for capturing a second image, said second image-capturing unit being wrapped in the endotracheal tube; and
at least one display for displaying said first image and second image.

2. The endotracheal tube installation system as claimed in claim 1, wherein the first image and the second image are transmitted to the display wirelessly.

3. The endotracheal tube installation system as claimed in claim 2, which has one display.

4. The endotracheal tube installation system as claimed in claim 1, which has one display.
